(19) United States
(12) Patent Application Publication
(10) Pub. No.: US 2021/0183495 A1
(43) Pub. Date: Jun. 17, 2021

(54) Functionalized Food as Medicine Platform and Computer System

(71) Applicant: Stephen Floe, Chanhassen, MN (US)

(72) Inventor: Stephen Floe, Chanhassen, MN (US)

(21) Appl. No.: 17/120,082

(22) Filed: Dec. 11, 2020

Related U.S. Application Data
(60) Provisional application No. 62/946,965, filed on Dec. 11, 2019.

(51) Int. Cl. G16H 20/60; G16H 10/60; G06Q 30/06; G06Q 20/20; G06Q 40/08

(52) U.S. Cl. CPC G16H 20/60; G16H 10/60; G06Q 40/08; G06Q 30/0643; G06Q 20/20; G06Q 30/0635

(57) ABSTRACT

This document generally describes technology related to medical and patient treatment technologies, including patient therapies provided and facilitated around food as medicine (“FAM”). In particular, this document describes technologies for functionalizing FAM therapies in patients by providing a platform to facilitate connections between partner systems, such as medical providers, insurers, clinically sound FAM therapies, and grocers/retailers, in a way that aligns incentives among the partner systems and that leads to patient compliance and success.

### CROSS-REFERENCE TO RELATED APPLICATIONS

This application claims the benefit of priority to U.S. Provisional Application No. 62/946,965, filed on Dec. 11, 2019, the entire content of which is hereby incorporated by reference.

### TECHNICAL FIELD

This document generally describes technology related to medical and patient treatment technologies, including patient therapies provided and facilitated around food as medicine (“FAM”).

### BACKGROUND

FAM is a currently an advancing area of medicine, with increasing bodies of evidence, supported by recognition that responding to disease via procedures or pharmaceuticals only after progression continues to prove costly, both economically and in terms of patient experience and outcomes. FAM aims to address causes of chronic disease for prevention as well as provide support for patients through specific, personalized guidance addressing conditions that have already progressed. Hypertension, diabetes, heart disease, and other conditions are highly relevant for FAM. FAM is supported by studies that have shown a reduction in emergency rooms visits, success in reversing type II diabetes, and other positive results from real world evidence. Research and further study is ongoing, but initial results present a compelling case to functionalize a process around established credible clinical evidence

### SUMMARY

This document generally describes technologies for functionalizing FAM therapies in patients by providing a platform to facilitate connections between partner systems, such as medical providers, insurers, clinically sound FAM therapies, and grocers/retailers, in a way that aligns incentives among the partner systems and that leads to patient compliance and success. One of the main challenges with FAM therapies is that these partner systems are disparate and disconnected, which results in patients bearing the brunt of following and complying with FAM therapy plans. For example, a patient who has been prescribed a specific FAM therapy that includes a low sodium diet may find it daunting to understand and compare sodium levels in products at a grocery store, and whether these products are sufficiently low in sodium, especially when combined with other ingredients in a meal, to satisfy the FAM therapy plan. This burden on the patient presents a barrier to compliance, and can often lead to poor patient outcomes.

The disclosed technology is generally directed to a system and platform that creates and aligns incentives and creates connections between various partner systems so as to incentivize and facilitate each partner system's involvement with and support of patients undergoing FAM therapies. For example, the disclosed technology can analyze patient records and diagnoses, and automatically identify relevant, clinically sound FAM therapies to provide to the patient that are specifically tailored to treat the patient's ailments. Additionally, the disclosed technology can identify incentives that are available from insurers for particular patients who comply with FAM therapies, and can make those incentives available to patients as an incentive for their adherence to a FAM therapy. The disclosed technology can also translate FAM therapies into specific, actionable product and/or recipe recommendations at grocers and retailers, along with information on available insurer-provide incentives that are available for taking those actions. And further, the disclosed technology can automatically and accurately determine whether users have complied with their FAM therapies, providing verification to insurers before incentives are provided to patients.

In one implementation, a system for providing functionalized FAM services includes a medical computer system configured to maintain medical data for a patient, wherein the medical data includes information that identifies one or more medical conditions that the patient has been diagnosed with by a physician. The system can further include a FAM computer system configured to maintain FAM information that identifies FAM therapies and a food provider computer system configured to manage distribution and sale of food items by a food provider. The system can additionally include a user computing device configured to provide a FAM user interface for the patient and a functionalized FAM computer system configured to provide functionalized FAM services for the patient. The functionalized FAM services can include obtaining the medical data for the patient from the medial computer system; identifying the one or more medical conditions for the patient based on the medical data; identifying one or more FAM therapies from among the FAM therapies for the patient based on the one or more medical conditions and the FAM information; selecting one or more food items from among the food items offered by the food provider in compliance with the one or more FAM therapies; transmitting FAM recommendation information identifying the one or more food items to the user computing device in association with the one or more FAM therapies, wherein the user computing device is configured to output the information identifying the one or more food items in the FAM user interface; receiving purchase information from the food provider computer system for the patient; determining, after transmitting the FAM recommendation information to the user computing device, that the patient purchased the one or more food items based on the purchase information; and transmitting FAM therapy compliance information indicating that the patient has adhered to the one or more FAM therapies for the one or more medical conditions diagnosed by the physician based, at least in part, on the determination that the patient purchased the one or more food items.

Such a system can optionally include one or more of the following features. The functionalized FAM computer system can further include a cross-system correlation database system that stores correlation data that correlates (i) the one or more medical conditions to the one or more FAM therapies, (ii) the one or more FAM therapies to the one or more food items, and (iii) the one or more food items to the FAM therapy compliance information. Identifying the one or more FAM therapies from among the FAM therapies for the patient can be further based on the correlation data stored in the cross-system correlation database system. Selecting the one or more food items from among the food items offered by the food provider in compliance with the one or more FAM therapies can be further based on the correlation data stored in the cross-system correlation database system. Determining the FAM therapy compliance information can be further based on the correlation data stored in the cross-system correlation database system.

FAM therapy compliance information can be transmitted to the medical computer system for inclusion with the medical data for the patient. The medical computer system can be further configured to store the FAM therapy compliance information as part of the medical data for the patient and to make the FAM therapy compliance information available to the physician for the patient. The FAM therapy compliance information can be transmitted to the user computing device. The user computing device can be further configured to present the FAM therapy compliance information in the FAM user interface. The FAM recommendation information can include one or more recipes that adhere to the one or more FAM therapies and that use the one or more food items as ingredients. The user computing device can further be configured to output the one or more recipes in the FAM user interface. The user computing device can be further configured to prompt the user for digital evidence that the patient prepared the one or more recipes in the FAM user interface, record the digital evidence using one or more input devices that are part of the user computing device, and transmit the digital evidence to the functionalized FAM computer system. The functionalized FAM services can further include receiving the digital evidence from the user computing device, and determining the FAM therapy compliance information based, at least in part on, the digital evidence and the determination that the patient purchased the one or more food items. The digital evidence can include a digital image or a digital video taken by the user computing device. The determination of the FAM therapy compliance information can include determining, through comparison of the digital image or the digital video against one or more image-based models for the one or more recipes, a probability that the patient prepared the one or more recipes.

The system can further include an insurer computer system configured to provide incentives for compliance with the one or more FAM therapies by the patient. The FAM recommendation information can further include one or more incentives for the patient to consume the one or more food items to adhere with the one or more FAM therapies. The functionalized FAM services can further include selecting the one or more incentives from among the incentives provided by the insurer computer system based on the one or more FAM therapies for the patient. The FAM therapy compliance information can be transmitted to the insurer computer system in association with the one or more incentives. The insurer computer system can be further configured to award the one or more incentives to the patient based, at least in part, on the FAM therapy compliance information indicating that the patient has adhered to the one or more FAM therapies for the one or more medical conditions diagnosed by the physician.

The user computing device can be further configured to receive a user token from the functionalized FAM computer system that uniquely identifies the patient, present information to purchase the food items from the food provider in the FAM user interface, receive selection of the one or more food items in the FAM user interface, and transmit order information to the food provider computer system that identifies the one or more food items and the user token. The food provider computer system can be further configured to process the order information and, upon completion of a transaction for the one or more food items, transmit the purchase information with the user token to the functionalized FAM computer system. The functionalized FAM computer system can be further configured to determine that the patient purchased the one or more food items based on receipt of the user token with the purchase information. The user computing device can be further configured to transmit the user token to the food provider computer system as part of a query to present the information to purchase the food items, receive the information to purchase the food items from the food provider computer system customized for the patient and the one or more FAM therapies, wherein the information to purchase the food items is received with additional information identifying the one or more food items as being compliant with the one or more FAM therapies for the patient, and present the additional information in the FAM user interface to identify the one or more food items as being compliant with the one or more FAM therapies to the patient.

The food provider computer system can be further configured to receive the user token from the user computing device as part of the query to present the information to purchase the food items, poll the functionalized FAM computer system, using the user token, for selection of the food items offered by the food provider that will adhere to one or more FAM therapies, receive, from the functionalized FAM computer system, identification of the one or more food items and transmit the information to purchase the food items to the food provider application with the additional information identifying the one or more food items as being compliant with the one or more FAM therapies for the patient. The user token can be anonymized to the food provider computer system without any information that identifies the patient or the one or more FAM therapies for the patient to the food provider computer system.

The additional information can be presented in the FAM user interface with (i) graphical information identifying the one or more food items as being compliant with the one or more FAM therapies to the patient and (ii) one or more incentives for purchasing the one or more food items based on their compliance with the one or more FAM therapies for the patient. The incentives can be specific to the patient and presentations of the one or more food items to other users do not include the incentives. The additional information can be further presented in the FAM user interface with recipes that use the one or more food items to make one or more dishes that are compliant with the one or more FAM therapies. The system can further include an insurer computer system configured to provide incentives for compliance with the one or more FAM therapies by the patient. The one or more incentives can be selected from among the incentives provided by the insurer computer system based on the one or more FAM therapies for the patient.

The food provider can include a grocer or a retailer, and the food provider computer system comprises a point of sale system for the grocer or the retailer. The medical data can include electronic medical records.

The details of one or more implementations are set forth in the accompanying drawings and the description below. Other features and advantages will be apparent from the description and drawings, and from the claims.

### BRIEF DESCRIPTION OF THE DRAWINGS

FIG. 1 is a conceptual diagram of an example system providing functionalized FAM therapies to patients.

FIG. 2 is a block diagram of an example system for providing functionalized FAM therapy services to patients.

FIG. 3 is a block diagram of an example system for exchanging information among an example functionalized FAM computer system and a plurality of partner systems to provide functionalized FAM therapy services to patients.

FIG. 4 is an example technique for providing functionalized FAM therapy services to patients.

FIG. 5 is a conceptual diagram of an example system for providing FAM therapy guidance to a patient in compliance with a patient's personalized FAM therapy.

FIG. 6 is an example technique for providing functionalized FAM therapy services to patients.

FIGS. 7A-B are conceptual diagrams of example systems for providing FAM therapy guidance to a patient in compliance with a patient's personalized FAM therapy.

FIGS. 8A-B are screenshots of an example user interface on a client computing device for providing FAM therapy guidance to a patient through individual product guidance.

FIGS. 9A-B are screenshots of an example user interface on a client computing device for providing FAM therapy guidance to a patient through recipe-based product guidance.

FIG. 10 is a conceptual diagram of an example system for determining whether a patient has complied with FAM therapy and whether incentives can be awarded to the patient.

FIG. 11 is a conceptual diagram of an example system for determining whether a patient has complied with FAM therapy and whether incentives can be awarded to the patient.

FIG. 12 is a block diagram of computing devices that may be used to implement the systems and methods described in this document, as either a client or as a server or plurality of servers

### DETAILED DESCRIPTION OF ILLUSTRATIVE EMBODIMENTS

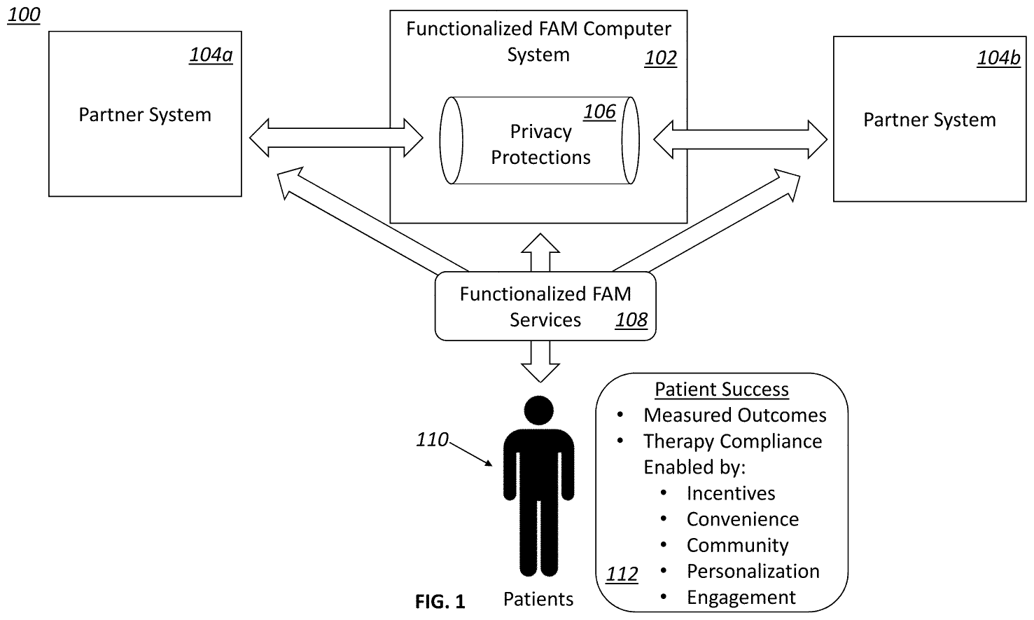

FIG. 1 is a conceptual diagram of an example system 100 providing functionalized FAM therapies to patients. The example system includes an example functionalized FAM computer system 102 (e.g., cloud computer system) that connects and facilitates interactions among a plurality of partner systems 104a-b, such as medical provider systems, care management systems, FAM therapy systems, insurers, grocers/retailers, and/or others. The functionalized FAM computer system 102 facilitates these connections with privacy protections 106 in place so that proprietary information from each partner system 104a-b is not compromised or shared with other systems, while at the same point sharing sufficient information so as to provide functionalized FAM services 108 to patients 110.

The privacy protections 106 can include, for example, generating global patient identifiers that are specific to the functionalized FAM computer system 102 and that link up user identifiers across the partner systems 104a-b in a way that de-identifies these partner-specific user identifiers in communication with the other partner systems. Additionally, the privacy protections 106 can include filtering, de-identifying, and/or obfuscating partner and/or patient information that is shared across the partner systems 104a-b. For example, information on a FAM therapy for a particular patient may be provided to a grocer/retailer with sufficient detail so that the grocer/retailer can provide FAM therapy guidance to the patient without identifying the underlying medical condition to which the FAM therapy is being applied.

The functionalized FAM computer system 102 can assist each of these partner systems 104a-b—medical providers, insurers, grocers/retailers, and/or others—in this process of providing FAM therapy services 108 to the patient 110 so that each partner system 104a-b is incentivized to participate. Additionally, the FAM therapy services 108 can be provided to the patient 110 in a way that it will have a high likelihood of success, including measuring outcomes and enabling therapy compliance through incentives being provided for the patient 110 to comply, convenience, providing community for the patient 110, personalizing the user experience, and/or providing an engaging platform for the patient 110.

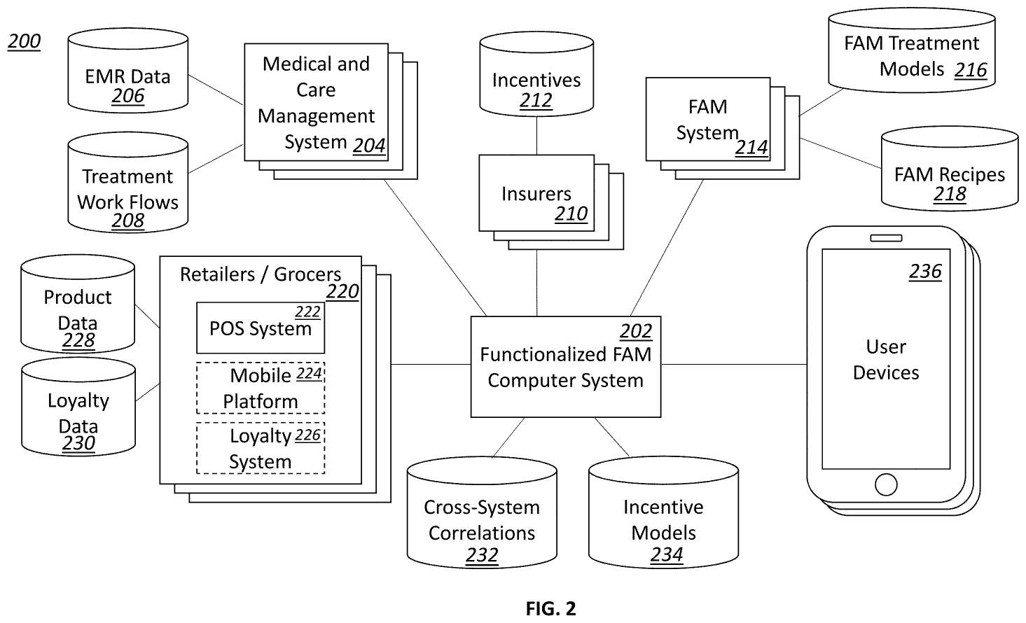

FIG. 2 is a block diagram of an example system 200 for providing functionalized FAM therapy services to patients. The system 200 can be similar to the system 100 described above.

The example system includes a functionalized FAM computer system 202 (similar to the system 102) that is connected over one or more networks to a plurality of partner systems, including medical and care management systems 204, insurers 2010, FAM systems 214, and retailers/grocers 220. The medical and care management systems 204 can be systems that are providing medical services, such as services provided by clinicians, and/or follow-up care management to patients. The medical and care management systems 204 can store a variety of medical data for patients, such as EMR data 206, and can also store treatment workflows 208, which outline workflows for treating various ailments that are diagnosed in patients. The medical and care management system 204 can share medical information for patients, such as medical history and diagnoses, with the functionalized FAM computer system 202, which can include clinician and/or medical provider requests for FAM therapies to be provided to patients.

The insurers 210 can provide health insurance services to patients, and can include FAM-related incentives 212 that they are willing to provide to patients who are willing and able to comply with FAM therapies. The FAM-related incentives 212 can include, for example, discounts and/or reimbursements that are provided to patients who comply with particular FAM therapies to treat their medical conditions.

The FAM system 214 can include systems that provide clinically proven FAM treatment models 216 for various medical conditions. The FAM treatment models 216 can identify types of foods to be consumed and/or avoided by patients with particular medical conditions, quantities of foods to be consumed, minimums and/or limits to foods to be consumed, and/or other dietary restrictions or requirements. The FAM treatment models 216 can identify particular FAM recipes 218 that comply with various FAM treatment models.

The retailers/grocers 220 can provide unprepared, partially prepared, and/or fully prepared food items for purchase by patients. The retailers/grocers 220 can include product data 228, which includes information on particular food items that they sell, as well as loyalty data 230 that details purchase histories for particular users. The retailers/grocers 220 can include point of sale (“POS”) systems 222, and in some instances mobile platforms 224 to interface with mobile apps specific to their systems and/or loyalty systems 226.

The functionalized FAM computer system 202 can include cross-system correlations 232 that correlate information provided from across the plurality of partner systems, such as correlating user identifiers from the medical and care management systems 204 with user identifiers that are used by the insurers 210 and the retailers/grocers 220. The functionalized FAM computer system 202 can also include incentive models 234 that model when and how to provide incentives provided by insurers 210 to patients, such as through the user devices 236 (e.g., mobile computing devices, non-mobile computing devices, wearable computing devices).

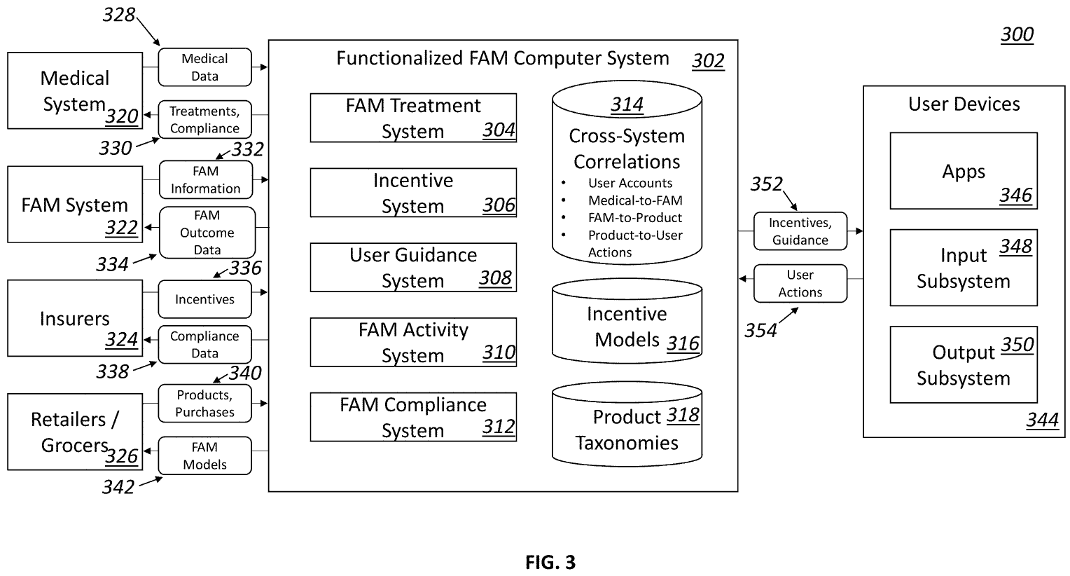

FIG. 3 is a block diagram of an example system 300 for exchanging information among an example functionalized FAM computer system 320 and a plurality of partner systems to provide functionalized FAM therapy services to patients. The system 300 can be similar to the systems 100 and/or 200 described above.

The functionalized FAM computer system 302 can receive medical data 328 from an example medical system 320 (similar to the medical system 204), which can include patient diagnoses and/or requests for FAM therapy services to be provided to patients. The functionalized FAM computer system 302 includes a FAM treatment system 304 that can map the medical data 328, and the patient diagnoses, to specific FAM therapies that are provided by the FAM system 322 (similar to the FAM system 214) via the FAM information 332, which can include FAM therapies, FAM recipes, and/or other FAM therapy details. The FAM treatment system 304 can use a cross-system correlation database 314 (similar to the cross-system correlations 232) to identify an appropriate FAM therapy to administer to a patient based on the medical data 328 for the patient.

The functionalized FAM computer system 302 further includes an incentive system 306 that can translate the FAM therapies identified by the FAM treatment system 304 into specific, actionable incentives that are available to be provided to particular patients undergoing FAM treatments based on available incentives 336 (similar to incentives 212) that are provided by the insurers 324 (similar to insurers 210). The incentive system 306 can use information stored in the cross-system correlations 314 and incentive models 316 to make these determinations.

The functionalized FAM computer system 302 additionally includes a user guidance system 308 that uses the determined FAM therapies for individual patients to identify specific products and/or recipes to product information 340 from retailers/grocers 326 (similar to retailers/grocers 220) to provide FAM therapy guidance to patients, such as generating specific product and/or recipe suggestions to be provided to patients. The user guidance system 308 can additionally provide guidance using the cross-system correlations 314, which can include mappings between FAM therapies and specific products, and using product taxonomies 318, which can provide hierarchical and logical groupings of products across a variety of characteristics, including food type, nutrition information, ingredients, and/or other information relevant to administering FAM therapies. The user guidance system 308 can additionally make guidance determinations based on product information 340 provided by retailers/grocers 326, such as information on specific products that are offered for sale by their establishments (e.g., product SKU information, product description, product details).

The incentives generated by the incentive system 306 and the guidance generated by the user guidance system 308 can be transmitted (352) to user devices 344 (e.g., mobile computing devices, such as smartphones), which can include apps 346 (e.g., mobile apps), input subsystems 348 (e.g., touchscreen, microphones, keyboard, sensors, cameras), and/or output subsystems 350 (e.g., display, speaker, tactile feedback systems). The user devices 344 can output the incentives and guidance to the user via, for example, the apps 346, such as web browsers, native mobile apps, and/or other applications that are suitable for providing a user interface through which information can be presented to the user and relevant user input can be received.

Information identifying user actions 354 related to FAM therapy guidance and incentives that are performed on the user devices 344 can be provided by the user devices 344 to the functionalized FAM computer system 302, and used by the FAM activity system 310 and/or the FAM compliance system 312. The FAM activity system 310 can determine whether products related to FAM therapies have been purchased by a patient using information on the user actions 354 (e.g., user purchased product using app 346) and/or using product purchase information 340 that is provided by the retailers/grocers 326 (e.g., purchases made using customer loyalty card, which can be correlated to the user account using the cross-system correlations 314). The FAM compliance system 312 can determine whether the patient has actually complied with the FAM therapies using the purchased products (e.g., cooked a meal using the products purchased), and can determine whether the patient has earned incentives that were offered as part of the FAM therapy. The FAM compliance system 312 can use the user actions information 354 from user devices 344, which may be one of multiple different user devices that are associated with user (e.g., one user may be associated with a smartphone, a smart watch, smart appliances, a and smart speaker) and which may be different from the device that is presenting FAM guidance and incentives, to determine whether a user has complied with the FAM therapy. For instance, the user actions 354 can include a digital picture of a cooked meal that is captured by the user device 344, transmitted to the functionalized FAM computer system 302, and analyzed the FAM compliance system 312 to determine whether the user complied with the FAM therapy guidance and/or incentives (e.g., prepare FAM therapy meal).

The functionalized FAM computer system 302 can provide information back to the partner systems 320-326 as part of the FAM therapy. For example, the functionalized FAM computer system 302 can provide FAM treatment and compliance information 320 to the medical systems 320, which can be added to patient records and used by clinicians and other medical professionals to provide medical treatment to patients. Additionally, the medical systems 320 may track and follow-up with patients to further determine and assess patient outcomes under the functionalized FAM computer system 302. The functionalized FAM computer system 302 can also provide FAM outcome data 334 back to the FAM system 322, which may be anonymized before it is transmitted so it can be used by the FAM system 322 to improve upon FAM therapy models and/or FAM recipes that are provided by the system 322. The functionalized FAM computer system 302 can also provide compliance data 338 to the insurers 324, which can used the compliance data 338 (measure outcome data) to determine whether and to what extent to award incentives to patients. The functionalized FAM computer system 302 can also provide FAM treatment models to the retailers/grocers, which can use the information to provide some level of FAM product guidance based on de-identified and/or anonymized user data.

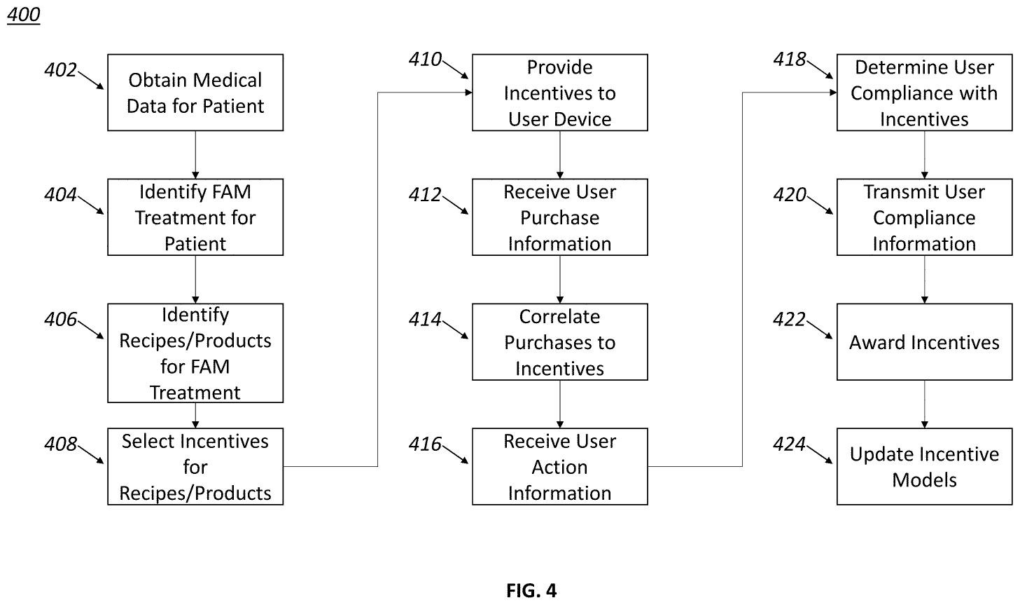

FIG. 4 is an example technique 400 for providing functionalized FAM therapy services to patients. The example technique 400 can be performed, for example, by the functionalized FAM computer system 102, 202, and/or 302, and/or other suitable computer systems and/or devices.

Medical data for a patient can be received (402) and used to identify a FAM treatment for the patient (404). Using the FAM treatment, recipes and products that are compliant with the FAM treatment can be identified (406) and, using that information, specific incentives (e.g., provided by insurers) for the FAM treatment products and/or recipes can be identified (408). Those incentives can be provided to and output by a user device (410), which may result in purchases of the incentives products and/or recipes by the user. Information identifying user purchases can be received (412), such as from the same user device that output the incentives, a different user device, and/or from a retailer/grocer system. Those purchases can be correlated to incentives provided to the user (414), such as by correlating a user identifier on a retailer/grocer platform (e.g., loyalty account identifier) with a user identifier for an insurer. User action information can be received after the purchase is made (416) and used to determine whether the user complied with the incentives using the purchased products (418). Such a determination can evaluate, for example, whether a user cooked a meal for their FAM treatment using the purchased products. The compliance determination can be transmitted (420), such as to an insurer, and used to award incentives to the user (422). The outcomes and results of the technique 400 can be used to update incentive models (424), such as through updating machine learning models that are used to identify, select, and provide incentives to users based on various criteria.

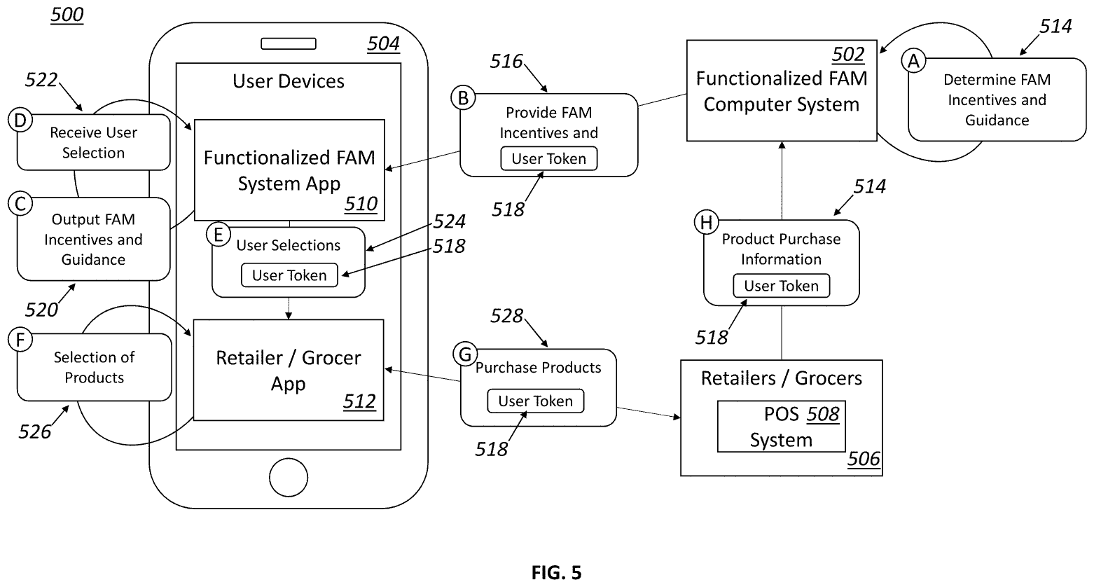

FIG. 5 is a conceptual diagram of an example system 500 for providing FAM therapy guidance to a patient in compliance with a patient's personalized FAM therapy. The example system 500 includes a functionalized FAM computer system 502 (similar to the functionalized FAM computer systems 102, 202, and/or 302), a user device 504 (similar to the user devices 236 and/or 344), and retailers/grocers 506 (similar to the retailers/grocers 220 and/or 326). In this example system 500, the user device 504 includes a functionalized FAM system app 510 that provides FAM therapy incentives and guidance, which can be used to direct FAM product purchases on a separate retailer/grocer app 512, which relays confirmation of the purchases back to the functionalized FAM computer system 502.

The functionalized FAM computer system 502 determines FAM incentives and guidance for a particular patient (step A, 514), which are then provided to the user device 504 with a user token 518 that uniquely identifies the patient (step B, 516). The user token 518 can be an identifier that is specific to the patient and the functionalized FAM computer system 502, and can be different from user identifiers on other platforms/systems. The user token 518 can be, for example, a permanent identifier (e.g., username) and/or a temporary identifier that is periodically reassigned (e.g., session identifier). The user token 518 can be passed between systems and/or used to generate additional unique identifiers between systems that can permit for information specific to patients to be shared between systems in an anonymous and de-identifiable way.

The functionalized FAM app 510 can output the FAM incentives and guidance (step C, 520) and receive user selection of the incentives and/or guidance (step D, 522). The selections, and/or portions thereof, can be provided to the retailer/grocer app 512 (step E, 524) along with the user token 518. The retailer/grocer app 512 can receive user selections of specific products that are associated with the selected FAM incentives and/or guidance (step F, 526), which can result in product purchases with a retailer/grocer system 506 and its POS system 508 (step G, 528). The product purchases can include, for example, the user token 518 which is passed along as part of the purchase, and then relayed as part of the confirmation of the products that were purchased (step H, 514).

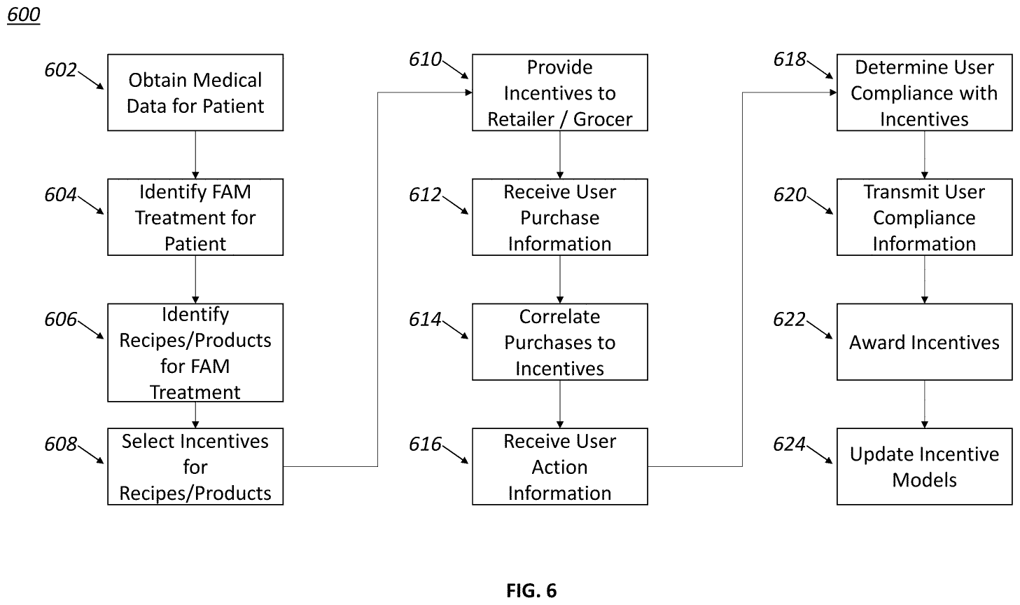

FIG. 6 is an example technique 600 for providing functionalized FAM therapy services to patients. The example technique 600 can be performed, for example, by the functionalized FAM computer system 102, 202, 302, and/or 502, and/or other suitable computer systems and/or devices.

Medical data for a patient can be received (602) and used to identify a FAM treatment for the patient (604). Using the FAM treatment, recipes and products that are compliant with the FAM treatment can be identified (606) and, using that information, specific incentives (e.g., provided by insurers) for the FAM treatment products and/or recipes can be identified (608). Those incentives can be provided to a retailer/grocer system along with a user identifier on the retailer/grocer system (610), which may use and present the incentives products and/or recipes to the user as part of a retailer/grocer-specific experience (e.g., retailer/grocer-specific app, retailer/grocer POS experience). Information identifying user purchases can be received (612), such as from the retailer/grocer system. Those purchases can be correlated to incentives provided to the retailer/grocer for a user (614), such as by correlating a user identifier on a retailer/grocer platform (e.g., loyalty account identifier) with a user identifier on the FAM computer system. User action information can be received after the purchase is made (616) and used to determine whether the user complied with the incentives using the purchased products (618). Such a determination can evaluate, for example, whether a user cooked a meal for their FAM treatment using the purchased products. The compliance determination can be transmitted (620), such as to an insurer, and used to award incentives to the user (622). The outcomes and results of the technique 600 can be used to update incentive models (624), such as through updating machine learning models that are used to identify, select, and provide incentives to users based on various criteria.

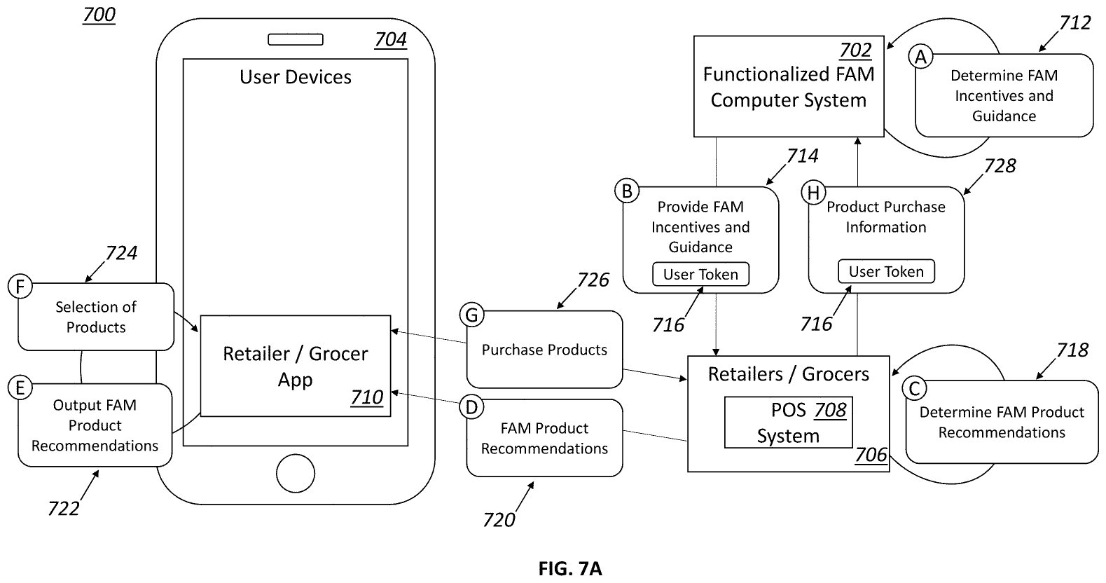

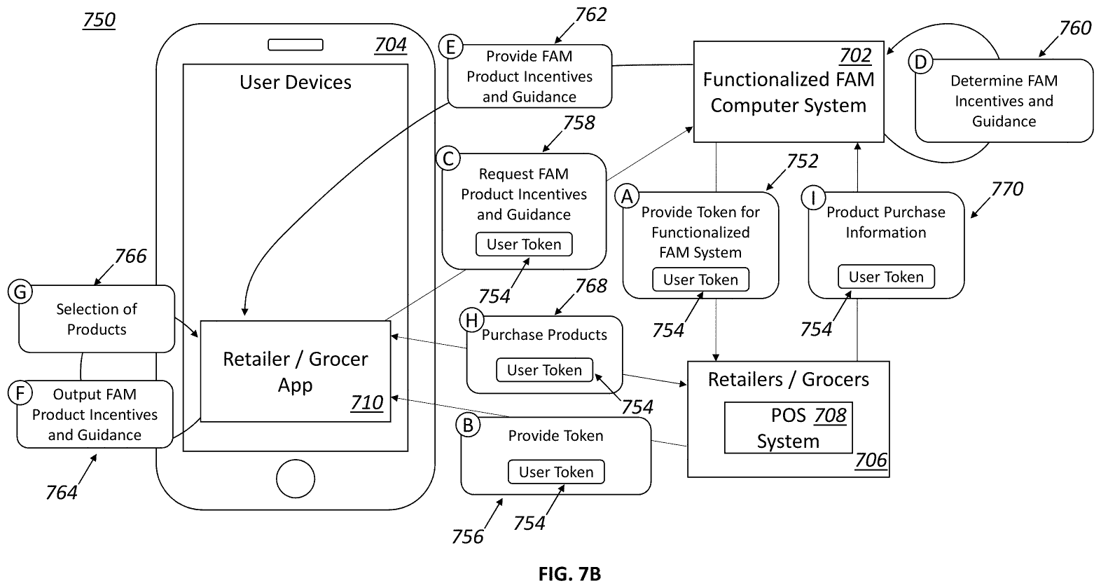

FIGS. 7A-B are conceptual diagrams of example systems 700 and 750 for providing FAM therapy guidance to a patient in compliance with a patient's personalized FAM therapy. The example systems 700 and 750 include a functionalized FAM computer system 702 (similar to the functionalized FAM computer systems 102, 202, 302, and/or 502), a user device 704 (similar to the user devices 236, 344, and/or 504), and retailers/grocers 706 (similar to the retailers/grocers 220, 326, and/or 506). In these example systems 700 and 750, the user device 704 includes a retailer/grocer app 710 through which FAM incentives and guidance are provided to the user. FIG. 7A depicts a system 700 in which the retailer/grocer system 706 receives some FAM therapy information for users (anonymized/de-identified in a way so that the retailer/grocer system 706 is unable to determine or infer a medical condition for the user) and makes local FAM product recommendations for the user. FIG. 7B depicts a system 750 in which the user device 704 polls the functionalized FAM computer system 702 for FAM product information, guidance, and incentives, which are output in the retailer/grocer app 710.

Referring to FIG. 7A, the functionalized FAM computer system 702 determined FAM incentives and guidance for a patient (step A, 712), which is provided to the retail/grocers 706 with a user token 716 (step B, 714). As discussed above with regard to FIG. 5, the user token 716 can anonymize/de-identify the patient in a way that the retailer/grocer 706 does not have user account information for other partner systems or access to other user information on those systems. The FAM incentives and guidance that are provided to the retailers/grocers 706 can additionally obfuscate and/or anonymize medical conditions that the patient may have that are being treated as part of the FAM incentives and guidance, such as simply providing information indicating some dietary parameters for the patient that could be indicative of any of a variety of medical conditions or of no medical conditions at all.

The retailers/grocers 706 can use the FAM incentives and/or guidance to determine specific FAM product recommendations to provide to the user device 704 that are compliant with the FAM incentives and/or guidance (step C, 718). In this instance, the retailers/grocers 706 may have some level of autonomy to select among and to prioritize among multiple different products, some of which may have promotions or other offers being provided by the retailers/grocers 706, to provide as the FAM product recommendations 718. For example, the retailer/grocer 706 may determine two types of rice may satisfy the FAM incentives and guidance for a patient, which permits the retailer/grocer 706 to select or prioritize the ranking of those two products to provide as the FAM product recommendations.

The FAM product recommendations can be provided to the retailer/grocer app 710 on the user device 704 (step D, 720), which can output the FAM product recommendations (step E, 722). The user can select products from the app 710 (step F, 724), which can result in purchases being processed with the retailers/grocers 706 and the POS system 708 (step G, 726). Once the product purchase is complete, the product purchase information can be transmitted back to the functionalized FAM computer system 702 along with the user token 716 so that the user action can be properly attributed to the user (step H, 728).

Referring to FIG. 7B, the functionalized FAM computer system 702 provides the user token 754 (similar to the user token 718) to the retailers/grocers 708 (step A, 752), which then forwards the user token 754 along to the retailer/grocer app 710 (step B, 756). When providing product information in the app 710, the retailer/grocer app 710 can poll the functionalized FAM computer system 702 for FAM product and incentive information that pertains to the products that are to be presented in the app 710 (step C, 758). For example, if the user selects to view breads (example product)that are sold by the retailer/grocer 706, the app 710 can retrieve the bread product offerings and then poll the functionalized FAM computer system 702 for FAM guidance and information that pertains to the bread product offerings. This polling can include transmitting the product information (e.g., SKUs, product identifiers) as well as the user token 754 so that the functionalized FAM computer system 702 can perform product-based FAM determinations for the patient. The FAM product incentives and guidance can be determined for the patient identified by the user token 754 and the products that are being polled (step D, 760), and the FAM product incentives and guidance can be provided back to the app 710 (step E, 762). The app 710 can output the produce and incentive guidance, along with the product information (step F, 764), which may result in user selections of the products (step G, 766) and purchase of the products (step H, 768). The purchases can be processed with the user token 754, which can be retransmitted to the functionalized FAM computer system 702 along with the purchase information (step I, 770).

Unlike system 700, the system 750 in FIG. 7B can restrict the distribution of FAM incentive and guidance information to the retailer/grocer 706 so that only that information that is relevant to specific products that are being presented. This can additionally protect user privacy, but can add additional steps for providing FAM information during the shopping/retail experience and can decrease the autonomy of retailers/grocers 706 to the determine and prioritize products that are offered on the user device 704.

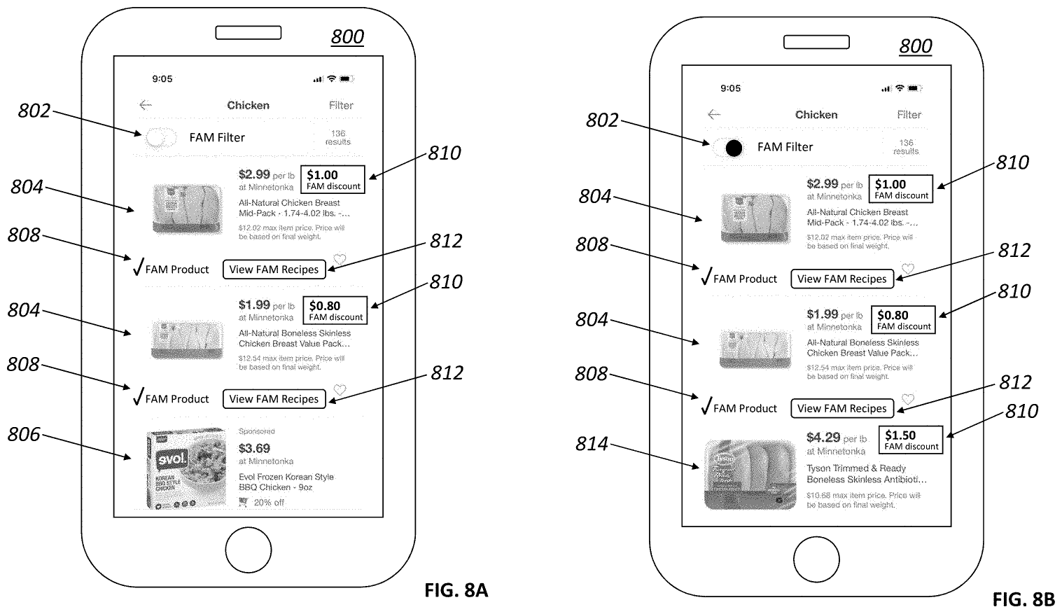

FIGS. 8A-B are screenshots of an example user interface on a client computing device 800 for providing FAM therapy guidance to a patient through individual product guidance. The user computing device 800 can be similar to the other user computing devices described throughout this document. The user interfaces that are depicted in FIGS. 8A-B can be presented, for example, as part of the systems 700 and 750, such as at steps 722 and 764.

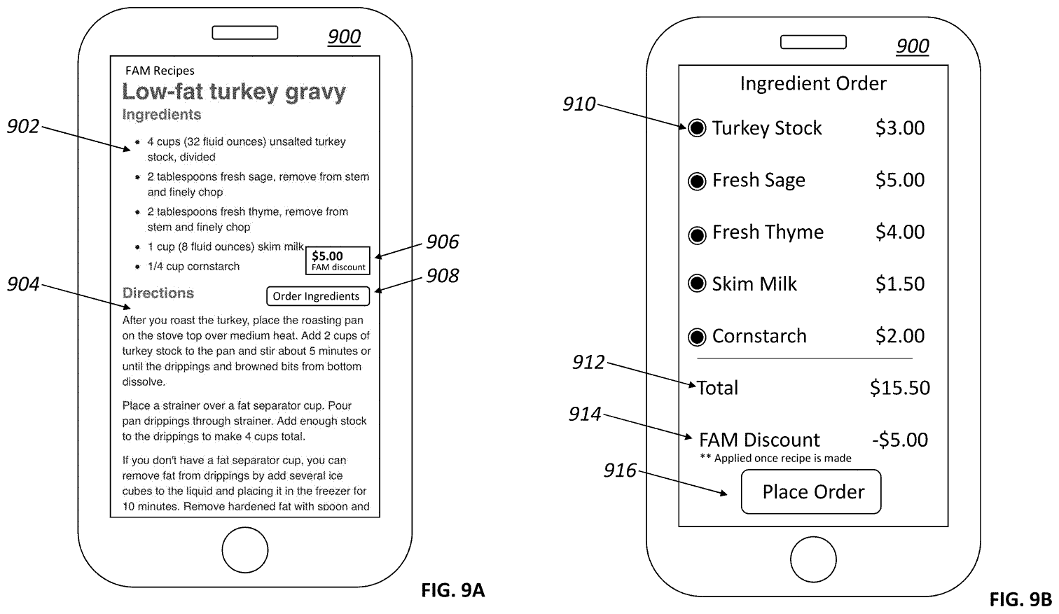

Referring to FIG. 8A, an example user interface is depicted showing chicken products 804-806 that are offered for sale by the retailer. The product offerings include FAM guidance information 808 providing confirmation that, for the user currently using the computing device 800, the particular products are part of their FAM therapy. The user interface additionally includes FAM guidance information 812 that provides a link to FAM recipes that use the products 804-805 are part of the user's FAM therapy (in this example the product 806 is not part of the FAM therapy). An example FAM recipe interface is described below with regard to FIGS. 9A-B. The user interface additionally include FAM incentive information 810 for each of the products 804 and 805, which provides discounts that will be provided to the user if the product is purchased and used as part of a FAM compliant meal. The user interface additionally includes a FAM filter 802 that the user can select in order to filter the view of products on the computing device 800 to only those products that are consistent and part of the user's FAM therapy. In this example, the FAM filter 802 is not being applied, so the product 806, which is not part of the FAM therapy, is presented.

Referring to FIG. 8B, the FAM filter 802 is selected, which causes the product 806 (example not part of the FAM therapy) to be hidden and replaced by the next FAM product 814 for the user in this category of products. In this view, the user interface presents only FAM products that are consistent with the user's FAM therapy. This FAM product 814 is also presented with the FAM incentive information 810, and the FAM guidance information (not shown).

FIGS. 9A-B are screenshots of an example user interface on a client computing device 900 for providing FAM therapy guidance to a patient through recipe-based product guidance. The user computing device 900 can be similar to the other user computing devices described throughout this document. The user interfaces that are depicted in FIGS. 9A-B can be presented, for example, as part of the systems 700 and 750, such as at steps 722 and 764.

Referring to FIG. 9A, the a FAM recipe for the user is presented with ingredients 902, directions 904, FAM incentive information 906, and a selectable interface element 908 (e.g., selectable button, link) through which the user can order all (or some) of the ingredients for the recipe.

Referring to FIG. 9B, in response to selection of the element 908, products 910 corresponding to the ingredients 902 (in sufficient quantities to satisfy the recipe requirements) are populated into an order (e.g., electronic shopping cart). The products 910 in this example are shown with prices and options to deselect one or more of the products, such as in instances where the user already has those ingredients at his/her house. The total 912 for the products to purchased, the FAM incentive 914 are shown, and a selectable element 916 to proceed with ordering the entire recipe are shown.

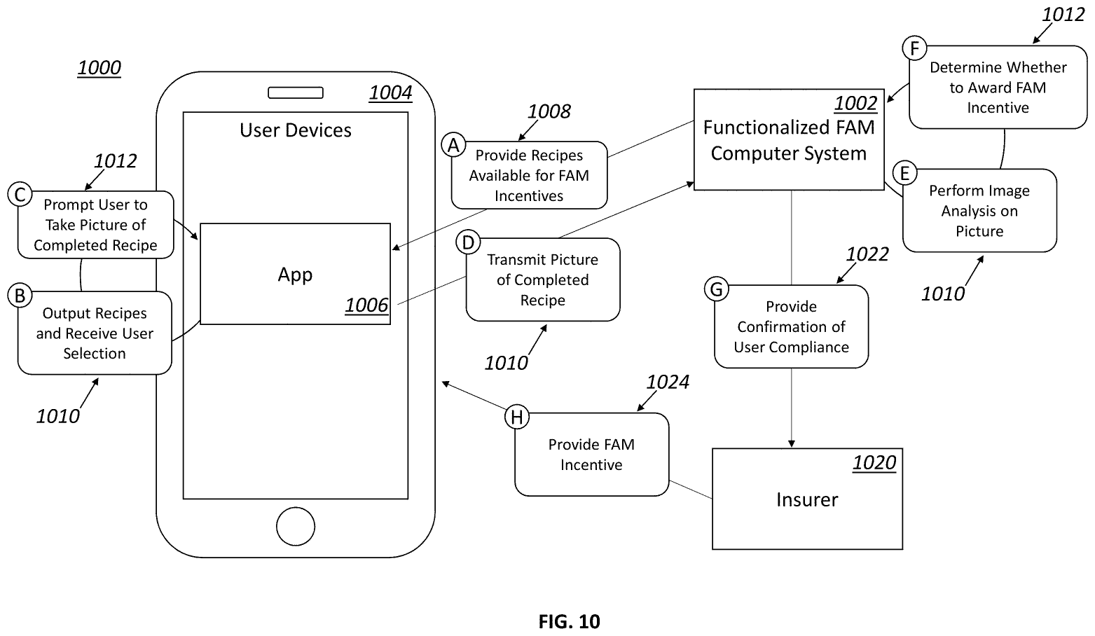

FIG. 10 is a conceptual diagram of an example system 1000 for determining whether a patient has complied with FAM therapy and whether incentives can be awarded to the patient. The example system 1000 includes a functionalized FAM computer system 1002 (similar to the functionalized FAM computer systems 102, 202, 302, 502, 702), a user device 1004 (similar to the user devices 236, 344, 504, 704), and insurer 1020 (similar to the insurers discussed above).

In this example, the functionalized computer system 1002 provides recipes that are available for FAM incentives to the user device 1004 (step A, 1008), which are output by the app 1006 and selected by the user (step B, 1010). Selection of an incentive can prompt the user to take a picture of the completed recipe (step C, 1012), in response to which the app 1006 can transmit the picture to the functionalized computer system 1002 (step D, 1014). The functionalized computer system 1002 can perform image analysis on the picture to determine whether the item depicted in the picture is likely to be the dish resulting from cooking the selected recipe with the incentive (step E, 1016). Any of a variety of techniques can be used, such as image comparison operations using machine learning models trained on a variety of different dishes cooked by a variety of different users and photographed in a variety of different environments. The functionalized computer system 1002 can determine, based on the image analysis, whether to award the FAM incentive for the user action (following the recipe), as indicated by the verifiable evidence (picture of the dish) (step F, 1018). If the functionalized computer system 1002 determines the user has complied with the FAM incentive, the functionalized computer system 1002 can transmit the confirmation to the insurer 1020 (step G, 1022), which can then provide the incentive to the user (step H, 1024).

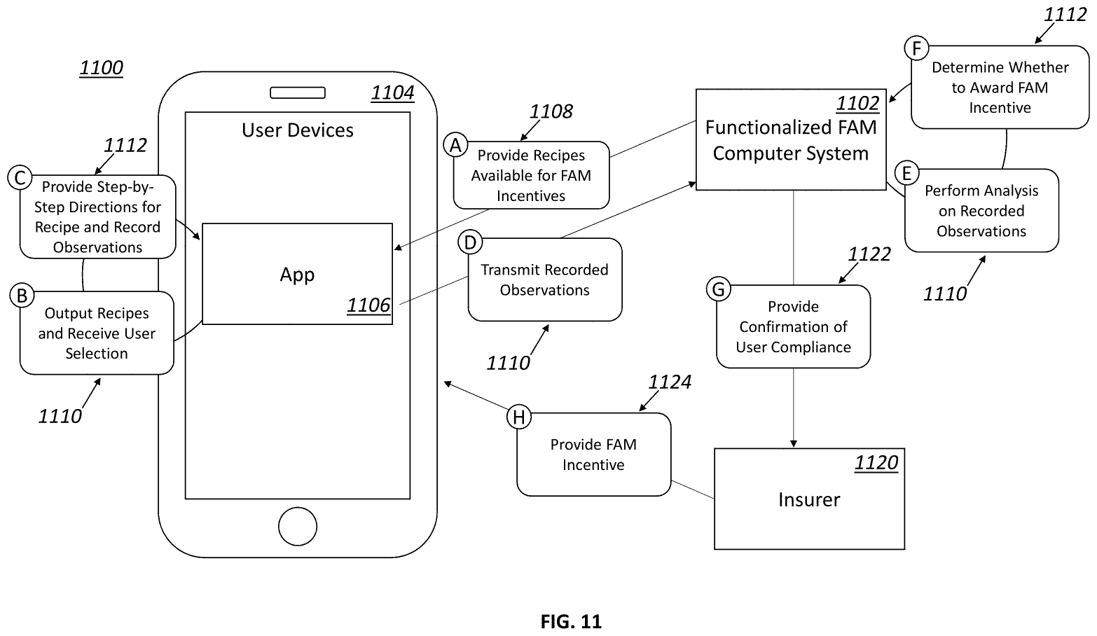

FIG. 11 is a conceptual diagram of an example system 1100 for determining whether a patient has complied with FAM therapy and whether incentives can be awarded to the patient. The example system 1100 includes a functionalized FAM computer system 1102 (similar to the functionalized FAM computer systems 102, 202, 302, 502, 702, 1102), a user device 1104 (similar to the user devices 236, 344, 504, 704, 1004), and insurer 1120 (similar to the insurers discussed above).

In this example, the functionalized computer system 1102 provides recipes that are available for FAM incentives to the user device 1104 (step A, 1108), which are output by the app 1106 and selected by the user (step B, 1110). Selection of a recipe with an incentive can prompt the app 1106 to provide step-by-step directions for performing the recipe, some of which may be confirmed and prompted by the user when the user is ready for the next step (step C, 1112). The app 1106 can record observations during the performance of the recipe by the user, such as timing between steps, ambient sounds, speech in the background, and/or other signals (step C, 1012). Upon completion of the step-by-step direction, the app 1006 can transmit the observations to the functionalized computer system 1102 (step D, 1114). The functionalized computer system 1102 can perform analysis on the recorded observations to determine whether the observations are consistent with the user having actually made recipe associated with the incentive (step E, 1116). Any of a variety of techniques can be used, such as evaluating timing between steps relative to a range of expected time intervals, the total duration of time taken to complete the recipe relative to a range of expected durations, ambient noises consistent with the physical actions required of the user to perform steps (e.g., whisk egg, blend products). The functionalized computer system 1102 can determine, based on the observation analysis, whether to award the FAM incentive for the user action (following the recipe), as indicated by the verifiable evidence (observations during the steps performed) (step F, 1118). If the functionalized computer system 1102 determines the user has complied with the FAM incentive, the functionalized computer system 10102 can transmit the confirmation to the insurer 1120 (step G, 1122), which can then provide the incentive to the user (step H, 1124).

Additionally and/or alternatively, the functionalized FAM computer systems described throughout this document can use ICD10 codes for social determinants to provide FAM-related services. For example, a patient who has had an ICD10 social determinant code designated in his/her medical records can receive specialized FAM therapies that are consistent with and treat the ICD10 code, which can be provided according to the systems, techniques, and devices described throughout this document.

Additionally and/or alternatively, the functionalized FAM computer systems described throughout this document can provide, as part of the FAM therapies, prepared and delivered meals to patients, such as to the elderly and/or other patients with mobility issues.

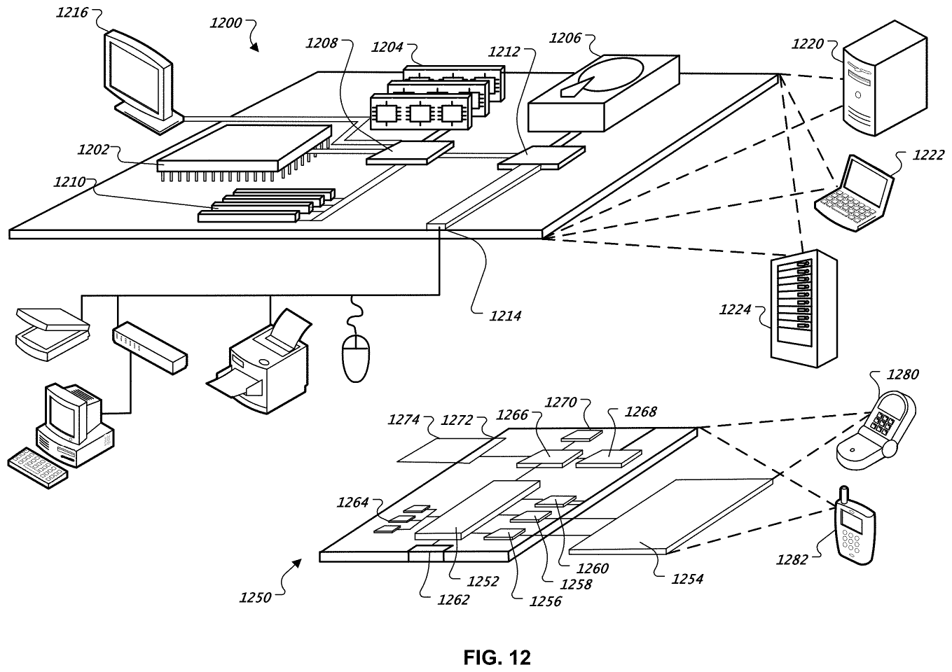

FIG. 12 is a block diagram of computing devices 1200, 1250 that may be used to implement the systems and methods described in this document, as either a client or as a server or plurality of servers. Computing device 1200 is intended to represent various forms of digital computers, such as laptops, desktops, workstations, personal digital assistants, servers, blade servers, mainframes, and other appropriate computers. Computing device 1250 is intended to represent various forms of mobile devices, such as personal digital assistants, cellular telephones, smartphones, and other similar computing devices. The components shown here, their connections and relationships, and their functions, are meant to be examples only, and are not meant to limit implementations described and/or claimed in this document.

Computing device 1200 includes a processor 1202, memory 1204, a storage device 1206, a high-speed interface 1208 connecting to memory 1204 and high-speed expansion ports 1210, and a low speed interface 1212 connecting to low speed bus 1214 and storage device 1206. Each of the components 1202, 1204, 1206, 1208, 1210, and 1212, are interconnected using various buses, and may be mounted on a common motherboard or in other manners as appropriate. The processor 1202 can process instructions for execution within the computing device 1200, including instructions stored in the memory 1204 or on the storage device 1206 to display graphical information for a GUI on an external input/output device, such as display 1216 coupled to high-speed interface 1208. In other implementations, multiple processors and/or multiple buses may be used, as appropriate, along with multiple memories and types of memory. Also, multiple computing devices 1200 may be connected, with each device providing portions of the necessary operations (e.g., as a server bank, a group of blade servers, or a multi-processor system).

The memory 1204 stores information within the computing device 1200. In one implementation, the memory 1204 is a volatile memory unit or units. In another implementation, the memory 1204 is a non-volatile memory unit or units. The memory 1204 may also be another form of computer-readable medium, such as a magnetic or optical disk.

The storage device 1206 is capable of providing mass storage for the computing device 1200. In one implementation, the storage device 1206 may be or contain a computer-readable medium, such as a floppy disk device, a hard disk device, an optical disk device, or a tape device, a flash memory or other similar solid state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 1204, the storage device 1206, or memory on processor 1202.

The high-speed controller 1208 manages bandwidth-intensive operations for the computing device 1200, while the low speed controller 1212 manages lower bandwidth-intensive operations. Such allocation of functions is an example only. In one implementation, the high-speed controller 1208 is coupled to memory 1204, display 1216 (e.g., through a graphics processor or accelerator), and to high-speed expansion ports 1210, which may accept various expansion cards (not shown). In the implementation, low-speed controller 1212 is coupled to storage device 1206 and low-speed expansion port 1214. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 1200 may be implemented in a number of different forms, as shown in the figure. For example, it may be implemented as a standard server 1220, or multiple times in a group of such servers. It may also be implemented as part of a rack server system 1224. In addition, it may be implemented in a personal computer such as a laptop computer 1222. Alternatively, components from computing device 1200 may be combined with other components in a mobile device (not shown), such as device 1250. Each of such devices may contain one or more of computing device 1200, 1250, and an entire system may be made up of multiple computing devices 1200, 1250 communicating with each other.

Computing device 1250 includes a processor 1252, memory 1264, an input/output device such as a display 1254, a communication interface 1266, and a transceiver 1268, among other components. The device 1250 may also be provided with a storage device, such as a microdrive or other device, to provide additional storage. Each of the components 1250, 1252, 1264, 1254, 1266, and 1268, are interconnected using various buses, and several of the components may be mounted on a common motherboard or in other manners as appropriate.

The processor 1252 can execute instructions within the computing device 1250, including instructions stored in the memory 1264. The processor may be implemented as a chipset of chips that include separate and multiple analog and digital processors. Additionally, the processor may be implemented using any of a number of architectures. For example, the processor may be a CISC (Complex Instruction Set Computers) processor, a RISC (Reduced Instruction Set Computer) processor, or a MISC (Minimal Instruction Set Computer) processor. The processor may provide, for example, for coordination of the other components of the device 1250, such as control of user interfaces, applications run by device 1250, and wireless communication by device 1250.

Processor 1252 may communicate with a user through control interface 1258 and display interface 1256 coupled to a display 1254. The display 1254 may be, for example, a TFT (Thin-Film-Transistor Liquid Crystal Display) display or an OLED (Organic Light Emitting Diode) display, or other appropriate display technology. The display interface 1256 may comprise appropriate circuitry for driving the display 1254 to present graphical and other information to a user. The control interface 1258 may receive commands from a user and convert them for submission to the processor 1252. In addition, an external interface 1262 may be provide in communication with processor 1252, so as to enable near area communication of device 1250 with other devices. External interface 1262 may provided, for example, for wired communication in some implementations, or for wireless communication in other implementations, and multiple interfaces may also be used.

The memory 1264 stores information within the computing device 1250. The memory 1264 can be implemented as one or more of a computer-readable medium or media, a volatile memory unit or units, or a non-volatile memory unit or units. Expansion memory 1274 may also be provided and connected to device 1250 through expansion interface 1272, which may include, for example, a SIMM (Single In Line Memory Module) card interface. Such expansion memory 1274 may provide extra storage space for device 1250, or may also store applications or other information for device 1250. Specifically, expansion memory 1274 may include instructions to carry out or supplement the processes described above, and may include secure information also. Thus, for example, expansion memory 1274 may be provide as a security module for device 1250, and may be programmed with instructions that permit secure use of device 1250. In addition, secure applications may be provided via the SIMM cards, along with additional information, such as placing identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 1264, expansion memory 1274, or memory on processor 1252 that may be received, for example, over transceiver 1268 or external interface 1262.

Device 1250 may communicate wirelessly through communication interface 1266, which may include digital signal processing circuitry where necessary. Communication interface 1266 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 1268. In addition, short-range communication may occur, such as using a Bluetooth, WiFi, or other such transceiver (not shown). In addition, GPS (Global Positioning System) receiver module 1270 may provide additional navigation- and location-related wireless data to device 1250, which may be used as appropriate by applications running on device 1250.

Device 1250 may also communicate audibly using audio codec 1260, which may receive spoken information from a user and convert it to usable digital information. Audio codec 1260 may likewise generate audible sound for a user, such as through a speaker, e.g., in a handset of device 1250. Such sound may include sound from voice telephone calls, may include recorded sound (e.g., voice messages, music files, etc.) and may also include sound generated by applications operating on device 1250.

The computing device 1250 may be implemented in a number of different forms, as shown in the figure. For example, it may be implemented as a cellular telephone 1280. It may also be implemented as part of a smartphone 1282, personal digital assistant, or other similar mobile device.

Additionally computing device 1200 or 1250 can include Universal Serial Bus (USB) flash drives. The USB flash drives may store operating systems and other applications. The USB flash drives can include input/output components, such as a wireless transmitter or USB connector that may be inserted into a USB port of another computing device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” “computer-readable medium” refers to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing system that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), peer-to-peer networks (having ad-hoc or static members), grid computing infrastructures, and the Internet.

The computing system can include clients and servers. A client and server are generally remote from each other and typically interact through a communication network. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A system for providing functionalized food as medicine (“FAM”) services, the system comprising:
a medical computer system configured to maintain medical data for a patient, wherein the medical data includes information that identifies one or more medical conditions that the patient has been diagnosed with by a physician;
a FAM computer system configured to maintain FAM information that identifies FAM therapies;
a food provider computer system configured to manage distribution and sale of food items by a food provider;
a user computing device configured to provide a FAM user interface for the patient; and
a functionalized FAM computer system configured to provide functionalized FAM services for the patient, wherein the functionalized FAM services include:
obtaining the medical data for the patient from the medial computer system;
identifying the one or more medical conditions for the patient based on the medical data;
identifying one or more FAM therapies from among the FAM therapies for the patient based on the one or more medical conditions and the FAM information;
selecting one or more food items from among the food items offered by the food provider in compliance with the one or more FAM therapies;
transmitting FAM recommendation information identifying the one or more food items to the user computing device in association with the one or more FAM therapies, wherein the user computing device is configured to output the information identifying the one or more food items in the FAM user interface;
receiving purchase information from the food provider computer system for the patient;
determining, after transmitting the FAM recommendation information to the user computing device, that the patient purchased the one or more food items based on the purchase information; and
transmitting FAM therapy compliance information indicating that the patient has adhered to the one or more FAM therapies for the one or more medical conditions diagnosed by the physician based, at least in part, on the determination that the patient purchased the one or more food items.

2. The system of claim 1, wherein:
the functionalized FAM computer system further comprises a cross-system correlation database system that stores correlation data that correlates (i) the one or more medical conditions to the one or more FAM therapies, (ii) the one or more FAM therapies to the one or more food items, and (iii) the one or more food items to the FAM therapy compliance information,
identifying the one or more FAM therapies from among the FAM therapies for the patient is further based on the correlation data stored in the cross-system correlation database system,
selecting the one or more food items from among the food items offered by the food provider in compliance with the one or more FAM therapies is further based on the correlation data stored in the cross-system correlation database system, and
determining the FAM therapy compliance information is further based on the correlation data stored in the cross-system correlation database system.

3. The system of claim 1, wherein:
the FAM therapy compliance information is transmitted to the medical computer system for inclusion with the medical data for the patient, and
the medical computer system is further configured to store the FAM therapy compliance information as part of the medical data for the patient and to make the FAM therapy compliance information available to the physician for the patient.

4. The system of claim 1, wherein:
the FAM therapy compliance information is transmitted to the user computing device, and
the user computing device is further configured to present the FAM therapy compliance information in the FAM user interface.

5. The system of claim 1, wherein:
the FAM recommendation information includes one or more recipes that adhere to the one or more FAM therapies and that use the one or more food items as ingredients, and
the user computing device is further configured to output the one or more recipes in the FAM user interface.

6. The system of claim 5, wherein:
the user computing device is further configured to:
prompt the user for digital evidence that the patient prepared the one or more recipes in the FAM user interface,
record the digital evidence using one or more input devices that are part of the user computing device, and
transmit the digital evidence to the functionalized FAM computer system; and
the functionalized FAM services further include:
receiving the digital evidence from the user computing device, and
determining the FAM therapy compliance information based, at least in part on, the digital evidence and the determination that the patient purchased the one or more food items.

7. The system of claim 6, wherein:
the digital evidence comprises a digital image or a digital video taken by the user computing device,
the determination of the FAM therapy compliance information includes determining, through comparison of the digital image or the digital video against one or more image-based models for the one or more recipes, a probability that the patient prepared the one or more recipes.

8. The system of claim 1, further comprising:
an insurer computer system configured to provide incentives for compliance with the one or more FAM therapies by the patient;
wherein:
the FAM recommendation information further includes one or more incentives for the patient to consume the one or more food items to adhere with the one or more FAM therapies.

9. The system of claim 8, wherein the functionalized FAM services further include:
selecting the one or more incentives from among the incentives provided by the insurer computer system based on the one or more FAM therapies for the patient.

10. The system of 9, wherein:
the FAM therapy compliance information is transmitted to the insurer computer system in association with the one or more incentives, and
the insurer computer system is further configured to award the one or more incentives to the patient based, at least in part, on the FAM therapy compliance information indicating that the patient has adhered to the one or more FAM therapies for the one or more medical conditions diagnosed by the physician.

11. The system of claim 1, wherein:
the user computing device is further configured to:
receive a user token from the functionalized FAM computer system that uniquely identifies the patient,
present information to purchase the food items from the food provider in the FAM user interface,
receive selection of the one or more food items in the FAM user interface, and
transmit order information to the food provider computer system that identifies the one or more food items and the user token,
the food provider computer system is further configured to process the order information and, upon completion of a transaction for the one or more food items, transmit the purchase information with the user token to the functionalized FAM computer system, and
the functionalized FAM computer system is further configured to determine that the patient purchased the one or more food items based on receipt of the user token with the purchase information.

12. The system of claim 11, wherein:
the user computing device is further configured to:
transmit the user token to the food provider computer system as part of a query to present the information to purchase the food items;
receive the information to purchase the food items from the food provider computer system customized for the patient and the one or more FAM therapies, wherein the information to purchase the food items is received with additional information identifying the one or more food items as being compliant with the one or more FAM therapies for the patient; and
present the additional information in the FAM user interface to identify the one or more food items as being compliant with the one or more FAM therapies to the patient.

13. The system of claim 12, wherein the food provider computer system is further configured to:
receive the user token from the user computing device as part of the query to present the information to purchase the food items;
poll the functionalized FAM computer system, using the user token, for selection of the food items offered by the food provider that will adhere to one or more FAM therapies;
receive, from the functionalized FAM computer system, identification of the one or more food items; and
transmit the information to purchase the food items to the food provider application with the additional information identifying the one or more food items as being compliant with the one or more FAM therapies for the patient.

14. The system of claim 13, wherein the user token is anonymized to the food provider computer system without any information that identifies the patient or the one or more FAM therapies for the patient to the food provider computer system.

15. The system of claim 12, wherein the additional information is presented in the FAM user interface with (i) graphical information identifying the one or more food items as being compliant with the one or more FAM therapies to the patient and (ii) one or more incentives for purchasing the one or more food items based on their compliance with the one or more FAM therapies for the patient.

16. The system of claim 15, wherein the incentives are specific to the patient and presentations of the one or more food items to other users do not include the incentives.

17. The system of claim 15, wherein the additional information is further presented in the FAM user interface with recipes that use the one or more food items to make one or more dishes that are compliant with the one or more FAM therapies.

18. The system of claim 15, further comprising:
an insurer computer system configured to provide incentives for compliance with the one or more FAM therapies by the patient;
wherein the one or more incentives from among the incentives provided by the insurer computer system based on the one or more FAM therapies for the patient.

19. The system of claim 1, wherein the food provider comprises a grocer or a retailer, and the food provider computer system comprises a point of sale system for the grocer or the retailer.

20. The system of claim 1, wherein the medical data comprises electronic medical records.
